(19) United States
(12) Patent Application Publication
(10) Pub. No.: US 2026/0355275 A1
(43) Pub. Date: Nov. 12, 2026

(54) PHARMACEUTICAL COMBINATION AND USES THEREOF

(71) Applicant: Feng-Chou (Michael) LEE, Taipei City (TW)

(72) Inventor: Feng-Chou (Michael) LEE, Taipei City (TW)

(21) Appl. No.: 19/198,553

(22) Filed: May 5, 2025

(51) Int. Cl. A61K 38/16; A61K 31/343; A61P 35/00

(52) U.S. Cl. CPC A61K 38/16; A61K 31/343; A61P 35/00

(57) ABSTRACT

The present disclosure provides a pharmaceutical combination, comprising Ganoderma immunomodulatory protein (GMI), a recombinant thereof or a fragment thereof, and salvianolic acid B or a pharmaceutical acceptable salt thereof. The present disclosure further provides uses of the pharmaceutical combination for treating cancer stem cells.

### FIELD OF THE INVENTION

The present disclosure relates to a pharmaceutical combination, and more particularly to a pharmaceutical combination and uses thereof.

### SEQUENCE LISTING INFORMATION

The instant application contains a Sequence Listing which has been submitted electronically in ASCII XML format under Rule ST.26 and is hereby incorporated by reference in its entirety. Said ASCII copy, created on May 5, 2025, is named “G4590-22200US_20250505_SeqListing.xml” and is 4,602 bytes in size.

### BACKGROUND OF THE INVENTION

Cancer stem cell (CSC) theory has been increasingly considered in cancer research. Resistance to multiple drugs (MDR) in cancer is considered primarily attributable to the resilience of CSCs. Consequently, extensive research has been conducted to explore the mechanisms underlying MDR in these cells.

It is considered that cancers are fueled by a subpopulation of self-renewing CSCs, which have the capacity to produce the various differentiated cell types that make up the tumor. CSCs have been identified and isolated from tumors in various systems, including the hematopoietic system, as well as in breast, lung, prostate, colon, brain, head and neck, and pancreatic cancers.

Current therapies have primarily targeted the bulk of tumor cells in order to reduce tumor size. However, many cancer cells exhibit limited proliferative potential, whereby the ability to shrink a tumor largely reflects the capacity to eliminate the cells. Treatments that achieve complete tumor regression may leave behind sufficient CSCs to enable tumor regrowth. Only therapies specifically aimed at targeting CSCs are likely to yield more lasting responses and potentially lead to a cure for metastatic tumors.

### SUMMARY OF THE INVENTION

The present disclosure provides a pharmaceutical combination, comprising Ganoderma immunomodulatory protein (GMI), a recombinant thereof or a fragment thereof; and salvianolic acid B or a pharmaceutical acceptable salt thereof.

The present disclosure further provides a method of inhibiting and/or killing CSCs in a subject in need thereof, comprising administering the pharmaceutical combination to the subject.

The present disclosure also provides a method of treating and/or preventing cancers associated with CSCs cancer and/or treating cancer that is metastatic, refractory to a chemotherapy or radiotherapy, inherently resistant to chemotherapy or has relapsed in the subject after an initial treatment in a subject in need thereof, comprising administering the pharmaceutical combination to the subject.

Embodiment 1: A pharmaceutical combination or composition or kit, comprising: Ganoderma immunomodulatory protein (GMI), a recombinant thereof or a fragment thereof; and salvianolic acid B or a pharmaceutical acceptable salt thereof.

Embodiment 2: The pharmaceutical combination or composition or kit of Embodiment 1, wherein the GMI, a recombinant thereof or a fragment thereof is derived from Ganoderma lucidum, Ganoderma tsugae, Ganoderma microsporum or Ganoderma sinensis.

Embodiment 3: The pharmaceutical combination or composition or kit of any of Embodiments 1 and 2, wherein the GMI, a recombinant thereof or a fragment thereof is derived from Ganoderma microsporum.

Embodiment 4: The pharmaceutical combination or composition or kit of any of Embodiments 1 to 3, wherein the GMI, a recombinant thereof or a fragment thereof comprises an amino acid sequence of SEQ ID NO: 3.

Embodiment 5: The pharmaceutical combination or composition or kit of any of Embodiments 1 to 4, wherein the recombinant of GMI comprises an amino acid sequence of SEQ ID NO: 4.

Embodiment 6: The pharmaceutical combination or composition or kit of any of Embodiments 1 to 5, wherein the fragment of GMI, comprises an amino acid sequence of SEQ ID NO: 1 or 2.

Embodiment 7: The pharmaceutical combination or composition or kit of any of Embodiments 1 to 6, further comprising at least one pharmaceutically acceptable carrier.

Embodiment 8: The pharmaceutical combination or composition or kit of any of Embodiments 1 to 7, which is in the form of a kit of parts for combined administration.

Embodiment 9: The pharmaceutical combination or composition or kit of any of Embodiments 1 to 8, which is in the form of a combination product or a pharmaceutical composition.

Embodiment 10: A method of inhibiting and/or killing treating cancer stem cells in a subject in need thereof, comprising administering the pharmaceutical combination or composition or kit of any of Embodiments 1 to 8 to the subject.

Embodiment 11: The method of Embodiment 10, wherein the cancer stem cells are selected from the group consisting of pancreatic cancer stem cells and glioblastoma cancer stem cells.

Embodiment 12: The method of any of Embodiments 10 and 11, wherein the cancer stem cells are selected from the group consisting of pancreatic cancer stem cells and glioblastoma cancer stem cells.

Embodiment 13: The method of any of any of Embodiments 10 to 12, wherein the pancreatic cancer stem cells are pancreatic cancer cells expressing at least one of CD133, CD24, CD44, DCLK1, CXCR4, ESA, Oct4, and ABCB1.

Embodiment 14: The method of any of Embodiments 10 to 13, wherein the glioblastoma cancer stem cells are glioblastoma cancer cells expressing at least one of CD133, CD90, CD44, L1CAM, A2B5, and GPD1.

Embodiment 15: The method of any of Embodiments 10 to 14, wherein the GMI, a recombinant thereof or a fragment thereof and salvianolic acid B or a pharmaceutical acceptable salt thereof are administered jointly or independently at the same time.

Embodiment 16: The method of any of Embodiments 10 to 15, wherein the GMI, a recombinant thereof or a fragment thereof and salvianolic acid B or a pharmaceutical acceptable salt thereof are administered separately within time intervals.

Embodiment 17: A method of treating and/or preventing cancers associated with CSCs cancer or treating cancer that is metastatic, refractory to a chemotherapy or radiotherapy, inherently resistant to chemotherapy or has relapsed in the subject after an initial treatment in a subject in need thereof, comprising administering the pharmaceutical combination of claim 1 to the subject.

Embodiment 18: The method of Embodiment 17, wherein the cancer is selected from breast cancers, head and neck cancer, lung cancer, ovarian cancer, pancreatic cancer, colorectal carcinoma, prostate cancer, liver cancer, melanoma, multiple myeloma, brain tumor, sarcomas, medulloblastoma, glioblastoma, leukemia, and metastatic, refractory or relapsed of any of the above cancer.

Embodiment 19: The method of any of Embodiments 17 and 18, wherein the pancreatic cancer comprises pancreatic cancer cells expressing at least one of CD133, CD24, CD44, DCLK1, CXCR4, ESA, Oct4 and ABCB1.

Embodiment 20: The method of any of Embodiments 17 to 19, wherein the glioblastoma cancer comprises glioblastoma cancer cells expressing at least one of CD133, CD90, CD44, L1CAM, A2B5, and GPD1.

Embodiment 21: The method of any of Embodiments 17 to 20, wherein the GMI, a recombinant thereof or a fragment thereof and salvianolic acid B or a pharmaceutical acceptable salt thereof are administered jointly or independently at the same time.

Embodiment 22: The method of any of Embodiments 17 to 21, wherein the GMI, a recombinant thereof or a fragment thereof and salvianolic acid B or a pharmaceutical acceptable salt thereof are administered separately within time intervals.

### BRIEF DESCRIPTION OF DRAWINGS

FIGS. 1A to 1C show the cytotoxicity effect of GMI and salvianolic acid B (SalB) on PBMC. FIG. 1A: GMI. FIG. 1B: SalB. FIG. 1C: GMI+SalB.

FIGS. 2A to 2C show the synergic effect of GMI and salvianolic acid B (SalB) on pancreatic cancer stem cell (PCSC). FIG. 2A: GMI. FIG. 2B: SalB. FIG. 2C: GMI+SalB.

FIGS. 3A to 3C show the synergic effect of GMI and salvianolic acid B (SalB) on glioblastoma cancer stem cell (GBCSC). FIG. 3A: GMI. FIG. 3B: SalB. FIG. 3C: GMI+SalB.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated for reference.

In this application, the use of the singular includes the plural, the article “a” or “an” means “at least one,” and the use of “or” means “and/or,” unless specifically stated otherwise.

The term “preventing” or “prevention” is recognized in the art, and when used in relation to a condition, it includes administering an agent to reduce the frequency or severity of or to delay the onset of symptoms of a medical condition in a subject, relative to a subject which does not receive the agent.

As interchangeably used herein, the terms “individual,” “subject,” “host,” and “patient,” refer to a mammal, including, but not limited to, murines (rats, mice), non-human primates, humans, canines, felines, ungulates (e.g., equines, bovines, ovines, porcines, caprines), etc.

As used herein, the term “effective amount” means an amount effective to achieve a particular biological result such as, but not limited to, biological results disclosed, described, or exemplified herein. Such results can include, but are not limited to, the treatment of cancer stem cells.

Cancer stem cells (CSCs) are capable of self-renewal and differentiation, which may be related to chemotherapy resistance. Cancer stem cell theory has garnered significant attention, since cancer stem cells can evade cytotoxic effects of chemotherapeutic drugs, possibly explaining the failure of chemotherapy. Cancer stem cells, like normal stem cells, typically remain in the GO phase of the cell cycle, such that they do not enter the cell cycle. Currently, most chemotherapeutic drugs used in clinical settings focus on inhibiting the cell cycle and cell proliferation. As a result, chemotherapy drugs can suppress the growth of non-stem cancer cells that are actively cycling but fail to kill cancer stem cells. Once chemotherapy ends, the surviving cancer stem cells can regenerate and proliferate, leading to cancer recurrence. Additionally, some literature suggests that chemotherapeutic drugs may promote the subsequent proliferation of cancer stem cells. Current research indicates that known cancer treatment drugs (chemotherapeutic drugs) or those that can treat non-stem cancer cells may be insufficient to treat or kill cancer stem cells.

The present disclosure surprisingly found that Ganoderma immunomodulatory protein and salvianolic acid B can kill cancer stem cells. Specifically, Ganoderma immunomodulatory protein and salvianolic acid B have synergic effect on cancer stem cells, such as pancreatic cancer stem cells and glioblastoma cancer stem cells.

Accordingly, the present disclosure provides a pharmaceutical combination, comprising Ganoderma immunomodulatory protein (GMI), a recombinant thereof or a fragment thereof; and salvianolic acid B or a pharmaceutical acceptable salt thereof.

Fungal immunomodulatory proteins (FIPs) are a group of proteins found in fungi, which are extensively studied for their immunomodulatory activity including activation of immune cells, leading to immune-mediated anti-allergic, anti-inflammatory, and anti-tumor effects. Ganoderma immunomodulatory protein (GMI) is one kind of FIP derived from Ganoderma, such as from Ganoderma lucidum, Ganoderma tsugae, Ganoderma microsporum or Ganoderma sinensis.

The preparation of the GMI, a recombinant thereof or a fragment thereof has been described in U.S. Pat. No. 7,601,808. Particularly, the Ganoderma immunomodulatory protein is referred to as GMI, the recombinant of Ganoderma immunomodulatory protein is referred to as reGMI, and the fragment of Ganoderma immunomodulatory protein is referred to as SEQ ID NOs: 2 and 3 in U.S. Pat. No. 7,601,808.

In one embodiment, the GMI, a recombinant thereof or a fragment thereof as described herein comprises an amino acid sequence of SEQ ID NO: 3. In one embodiment, the recombinant of GMI comprises an amino acid sequence of SEQ ID NO: 4. In one embodiment, the fragment of GMI comprises an amino acid sequence selected from the group consisting of SEQ ID NOs: 1 to 2.

The sequences of SEQ ID NO: 1 to 4 are listed as follows.

(SEQ ID NO: 1)
LAWNVK
(SEQ ID NO: 2)
DLGVRPSYAV
(SEQ ID NO: 3)
MSDTALIFTLAWNVKQLAFDYTPNWGRGRPSSFIDTV
TFPTVLTDKAYTYRVVVSGKDLGVRPSYAVESDGSQK
INFLEYNSGYGIADTNTIQVYVIDPDTGNNFIVAQWN
(SEQ ID NO: 4)
EAEAEFMSDTALIFTLAWNVKQLAFDYTPNWGRGRPS
SFIDTVTFPTVLTDKAYTYRVVVSGKDLGVRPSYAVE
SDGSQKINFLEYNSGYGIADTNTIQVYVIDPDTGNNF
IVAQWNYLEQKLISEEDLNSAVDHHHHHH

Salvianolic acids, water-soluble components produced by many species of the genus Salvia, mainly extracted from Salvia miltiorrhiza, are a group of polyphenolic acids consisting of several combinations of caffeic acid and danshensu (salvianic acid) through ester and enol bonds. Salvianolic acid B (SalB) constitutes three molecules of danshensu and one molecule of caffeic acid. SalB is the most abundant and bioactive member of the hydrophilic components in Danshen, and contains seven phenolic hydroxyls which have been found to be closely related to redox potentials and/or antioxidant activities.

In some embodiments of the present disclosure, the pharmaceutical combination may be in the form of a kit of parts for combined administration. Alternatively, the pharmaceutical combination may be in the form of a combination product or a pharmaceutical composition.

In some embodiments of the present disclosure, the pharmaceutical combination is used for treating cancer, especially for treating cancer stem cells. In some embodiments of the present disclosure the cancer stem cells are pancreatic cancer stem cells and/or glioblastoma cancer stem cells. The pancreatic cancer stem cells may be isolated from pancreatic cancer cells, and may express CD133, CD24, CD44, DCLK1, CXCR4, ESA, Oct4, and/or ABCB1. The glioblastoma cancer stem cells may be isolated from glioblastoma cancer cells, and may express CD133, CD90, CD44, L1CAM, A2B5, and/or GPD1.

In some embodiments of the present disclosure, the cancer is pancreatic cancer and/or glioblastoma cancer. The pancreatic cancer may include pancreatic cancer cells expressing CD133, CD24, CD44, DCLK1, CXCR4, ESA, Oct4, and/or ABCB1. The glioblastoma cancer may include glioblastoma cancer cells expressing CD133, CD90, CD44, L1CAM, A2B5, and/or GPD1.

In some embodiments of the present disclosure, the GMI, a recombinant thereof or a fragment thereof and salvianolic acid B or a pharmaceutical acceptable salt thereof may be administered jointly or independently at the same time, or separately within time intervals.

In certain embodiments, the combination of GMI and salvianolic acid B discussed herein may be administered concurrently as a single composition in a pharmaceutically acceptable carrier, or concurrently as separate compositions with each in a pharmaceutically acceptable carrier. In another embodiment, the combination of GMI and salvianolic acid B can be administered sequentially. For example, GMI and salvianolic acid B can be administered sequentially, such as GMI being administered first and salvianolic acid B second, or salvianolic acid B being administered first and GMI second. Furthermore, if more than one dose of the combination therapy is administered sequentially, the order of the sequential administration can be reversed or kept in the same order at each time point of administration, sequential administrations may be combined with concurrent administrations, or any combination thereof. For example, the first administration of a combination GMI and salvianolic acid B may be concurrent, the second administration may be sequential with GMI first and salvianolic acid B second, and the third administration may be sequential with salvianolic acid B first and GMI second, etc. Another representative dosing scheme may involve a first administration that is sequential with salvianolic acid B first and GMI second or GMI first and salvianolic acid B second, and subsequent administrations may be concurrent.

The dosage of the pharmaceutical combination suitable for use according to the present disclosure can be determined by one of ordinary skill on the basis of the disclosure. The medicament will contain an effective dosage (depending upon the route of administration and pharmacokinetics of the active agent) of suitable pharmaceutical carriers and excipients suitable for the particular route of administration of the formulation (i.e., oral, parenteral, topical or by inhalation). The pharmaceutical combination is mixed into the pharmaceutical composition by means of mixing, dissolving, granulating, dragee-making, emulsifying, encapsulating, entrapping or lyophilizing processes.

Each of the two components of the pharmaceutical combination of the present disclosure can be administered to a patient either alone or in pharmaceutical combinations where it is mixed with suitable carriers and excipients. The pharmaceutical combination of the present disclosure can be administered parenterally, such as by intravenous injection or infusion, intraperitoneal injection, subcutaneous injection, or intramuscular injection. The pharmaceutical combination of the present disclosure can be administered orally or rectally through appropriate formulation with carriers and excipients to form tablets, pills, capsules, liquids, gels, syrups, slurries, suspensions and the like. The pharmaceutical combination of the present disclosure can be administered topically, such as by skin patch. The pharmaceutical combination of the present disclosure can be formulated into topical creams, skin or mucosal patch, liquids or gels suitable to topical application to skin or mucosal membrane surfaces. The pharmaceutical combination of the present disclosure can be administered by inhaler to the respiratory tract for local or systemic treatment.

The data presented in the present disclosure, together with recent advances in CSC research, enable the present disclosure to offer a range of methods aimed at inhibiting CSCs or treating cancers containing CSCs specifically, as well as cancers more broadly. Accordingly, methods for inhibiting and/or killing CSCs by the combination of the present disclosure or treating and/or preventing cancers associated with CSCs with the combination of the present disclosure are also provided.

Cancers known to have CSCs are good candidates for such treatments, and include but are not limited to: various types of breast cancers, head and neck cancers, lung cancers, ovarian cancers, pancreatic cancers, colorectal carcinoma, prostate cancers, liver cancers, melanoma, multiple myeloma, brain tumors, sarcomas, medulloblastoma, glioblastoma, and leukemia. Many metastatic forms of these cancers, such as metastatic breast cancer, have also been shown to contain CSCs. In one aspect, the methods described herein can be applied to treat a cancer selected from this group. In one embodiment, the methods of the invention may be used to treat cancers including, but not limited to, lung cancer, breast cancer, cervical cancer, colorectal carcinoma, liver cancer, head and neck cancer, pancreatic cancer, gastric cancer, brain cancer, glioblastoma, and prostate cancer.

Since CSCs have been shown to play a fundamental role in tumorigenesis, cancer metastasis, and recurrence, the methods of the present disclosure can be applied to treat cancers that are metastatic, resistant to chemotherapy or radiotherapy, inherently chemotherapy-resistant, or have relapsed following initial treatment.

The following examples are given for illustrating the pharmaceutical combination of the present disclosure and the uses thereof, but are not intended to limit the scope of the present invention.

### Example 1

### 1.1 Cultivation of Human Pancreatic Cancer Stem Cells and Glioblastoma Cancer Stem Cells

Pancreatic cancer stem cells (PCSC) and glioblastoma cancer stem cells (GBCSC) were purchased from Celprogen (CA, USA). PCSC and GBCSC were seeded in 10 cm petri-dish with AIM-V medium (Thermo Fisher Scientific, MA, USA) containing 10% FBS. When reaching confluence, cells were detached by using 0.15% trypsin at 37 °C for 6 min and collected cells were passaged at the initial cell seeding density every 3 days. Cell morphology was photographed by using phase contrast microscope.

### 1.2 PBMC Preparation

Whole blood was collected into heparin-coated tube and centrifuged at 400×g for 20 min at room temperature. After centrifugation, plasma fraction was removed. Buffy coat and erythrocyte fractions were mixed with equal volume of PBS. 4 ml of ficoll-paque solution was placed into a new 15 ml conical tube and slowly and gently released into a conical tube containing ficoll-paque solution without disturbing ficoll interphase. The tube was centrifuged at 600×g for 20 min at room temperature with no brake. The layer of mononuclear cells was collected to a new 15 ml conical tube and washed with PBS 3 times. Finally, cells were resuspended in cell culture medium, and then seeded to 24-well plate (2×10⁵ cells/well).

### 1.3 CFSE Staining

PCSC and GBCSC were suspended in 1× Assay Buffer at a concentration of 1×10⁷ cells/ml. Equal volume of CFSE solution was added to the cell suspension and incubated at 37 °C for 15 minutes. After incubation, cells were washed three times by PBS. Finally, cells were resuspended in cell culture medium, and then seeded to 24-well plate (1×10⁵ cells/well).

### 1.4 Cytotoxicity Analysis

The cytotoxicity of GMI and SalB was evaluated by using 7-AAD staining. Briefly, PCSC/GBCSC (1×10⁵ cells/well) and PBMC (2×10⁵ cells/well) were seeded in 24-well plate in the presence of various concentration of GMI and SalB at 37 °C incubator for 48 h followed by 7-AAD staining (Pharmingen, CA, USA). The frequency of cell death was analyzed using flow cytometry (BD Accuri™ C6 Plus).

### 1.5 Statistical Analysis

Statistical data are presented as mean±SEM. Statistical analysis was performed by Student's t test for comparison between two groups with significance set at a p value of less than 0.05.

### Example 2

### 2.1 the Cytotoxicity Effect of GMI and Salvianolic Acid B on PBMC

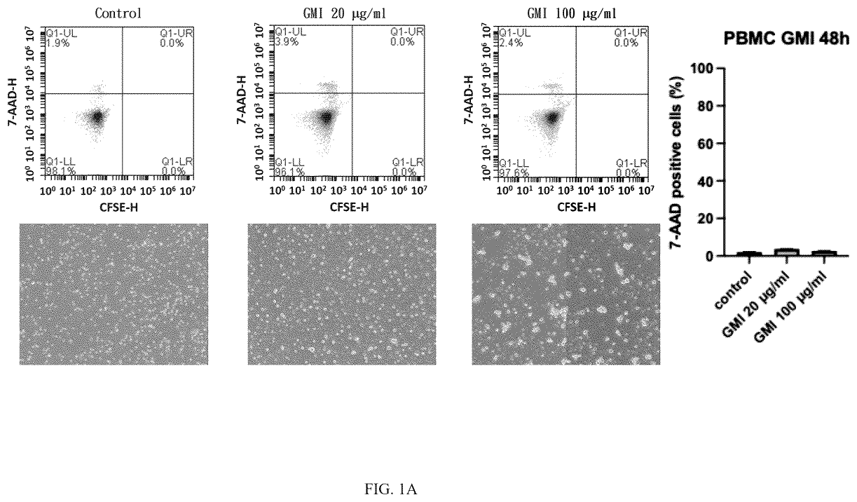

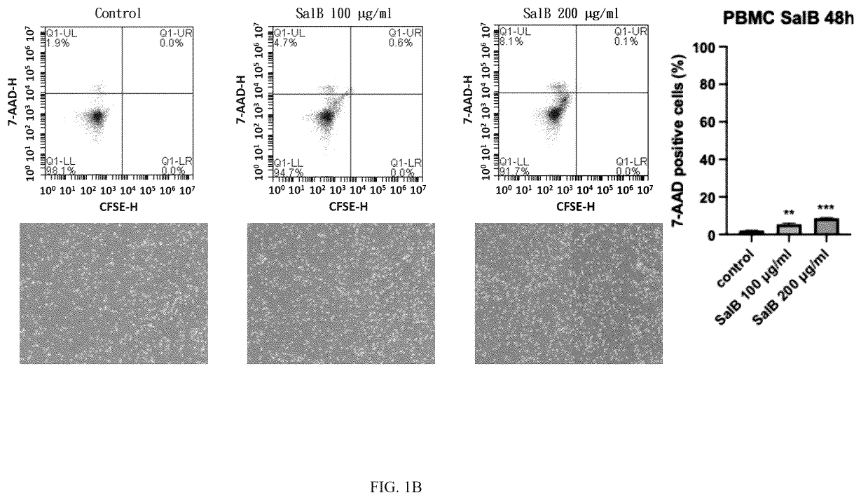

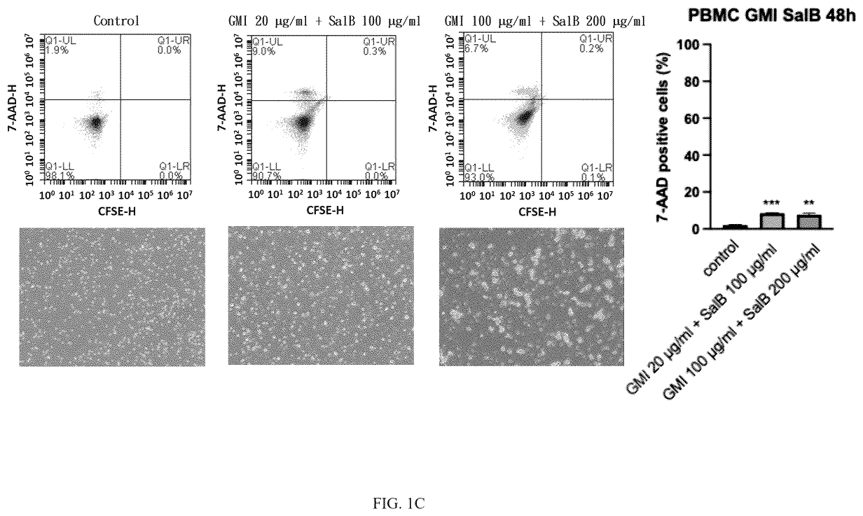

PBMC (2×10⁵ cells) was seeded into 24-well plate and then treated with different concentration of GMI and salvianolic acid B for 48 hours. After 7-AAD staining, the cytotoxicity effect of GMI and salvianolic acid B was further analyzed by flow cytometry. As shown in FIG. 1A, there is no significant difference between control and GMI-treated PBMC. Although salvianolic acid B could slightly induce cell death of PBMC, the frequency of cell death is lower than 10% (FIG. 1B). We also found that the frequency of cell death on PBMC was lower than 10% under GMI and salvianolic acid B treatment (FIG. 1C). Taken these results together, we demonstrated that GMI and salvianolic acid B treatment will not cause severe damage to PBMC.

### Example 3

### 3.1 the Synergic Effect of GMI and Salvianolic Acid B on Pancreatic Cancer Stem Cell

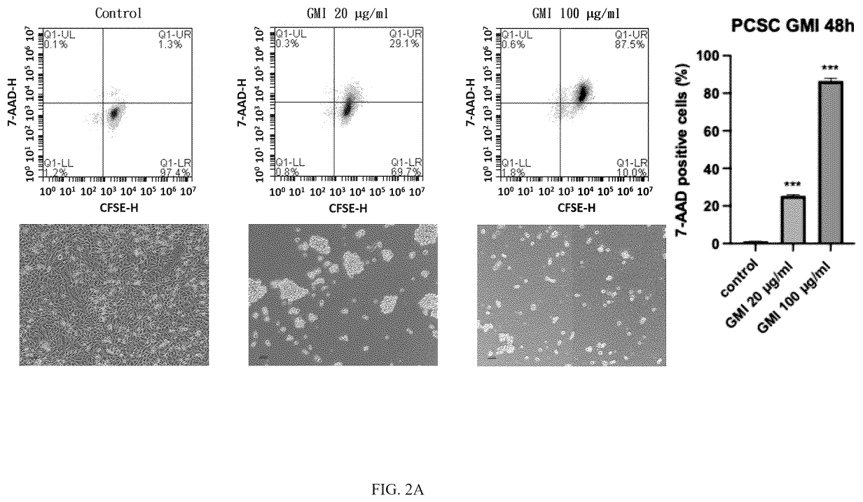

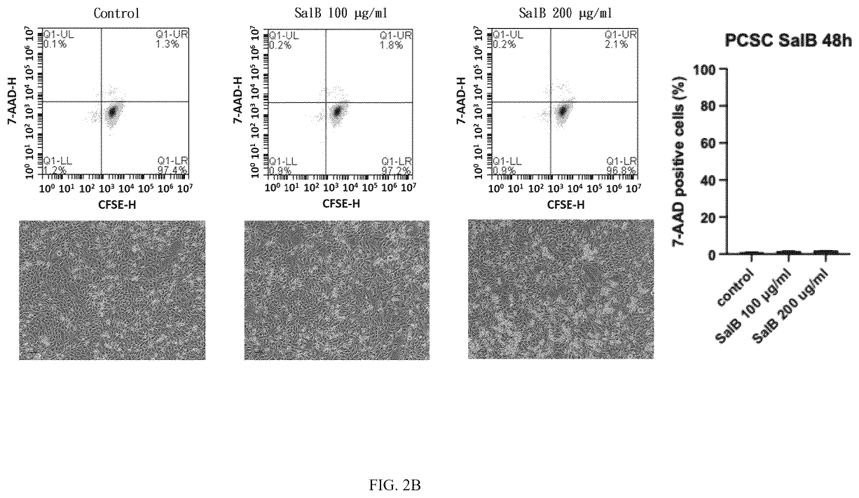

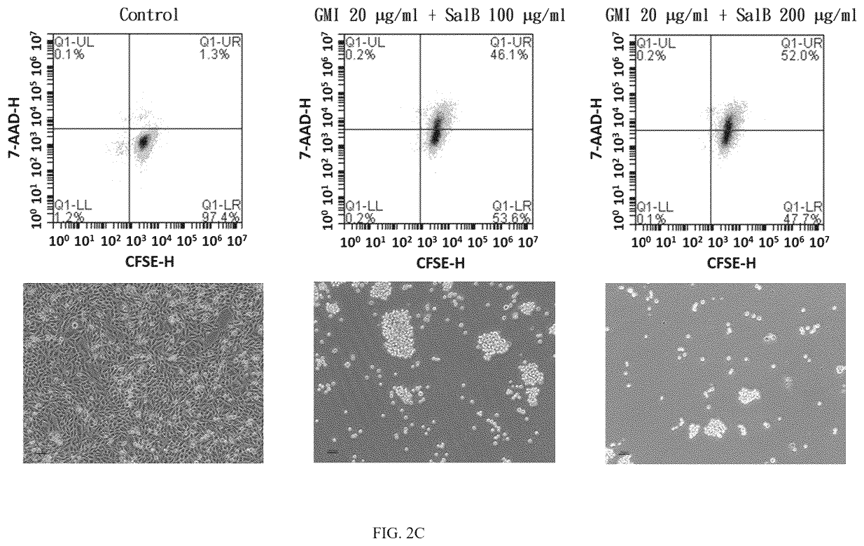

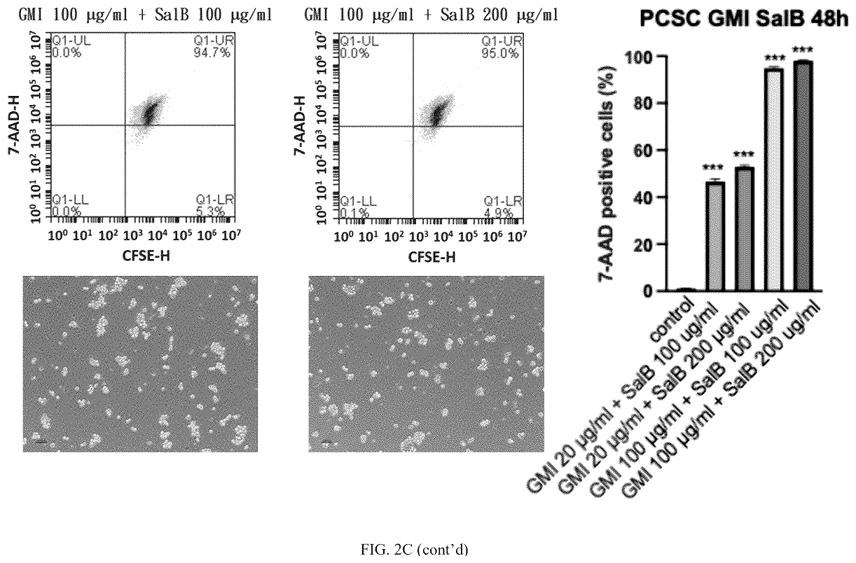

In order to mimic tumor microenvironment, we investigated the cytotoxicity effect of GMI and salvianolic acid B on pancreatic cancer stem cell (PCSC) under PBMC existed condition. First, PCSC (1×10⁵ cells) and PBMC (2×10⁵ cells) were seeded into 24-well plate and treated with different concentration of GMI or salvianolic acid B for 48 hours. After 7-AAD staining, the frequency of PCSC death was analyzed by flow cytometry. As shown in FIG. 2A, GMI treatment could induce cell death on PCSC. However, salvianolic acid B treatment has no effect on PCSC (FIG. 2B). Nevertheless, the sensitivity to GMI in PCSC was dramatically improved in combination with salvianolic acid B (FIG. 2C). Overall, these findings demonstrated the enhanced anti-PCSC activity via the synergic effect of GMI and salvianolic acid B.

3.2 the Synergism of GMI and Salvianolic Acid B of this Invention for PCSC Cytotoxicity

Three different effects of multiple drugs have been proposed, including (1) synergism; (2) additive effect and (3) antagonism. The combination index (CI) published by Chou TC. et al. Quantitative analysis of dose-effect relationships: the combined effects of multiple drugs or enzyme inhibitors, Adv Enzyme Regul, Vol. 22, 1984, pp. 27-55 and Liang Zhao et al. Evaluation of Combination Chemotherapy: Integration of Nonlinear Regression, Curve Shift, Isobologram, and Combination Index Analysis, Clinical Cancer Research, Vol. 10, 2004, pp. 7994-8004 can be used in analyzing the drug-drug interaction. The calculation formula is CI=C_{A,x}/IC_{x,A}+C_{B,x}/IC_{x,B}. Wherein, C_{A,x} and C_{B,x} are the concentrations of drug A and drug B used in combination to achieve x % drug effect. IC_{x,A} and IC_{x,B} are the concentrations for single agents to achieve x % drug effect. CI<1 indicates that the drugs used in combination have synergism. CI=1 indicates that the drugs used in combination have additive effect. CI>1 indicates that the drugs used in combination have antagonism.

In this example, the results of FIGS. 2A-2C which presented the data of the rates of cell cytotoxicity statistics from three independent experiments analyzed by the flow cytometer. As shown in FIG. 2A, the rates of cell cytotoxicity of the control group, GMI group (20 μg/mL), and GMI group (100 μg/mL) are 1.17%, 25.4%, and 86.53%, respectively. As shown in FIG. 2B, the rates of cell cytotoxicity of the Salvianolic acid B group (100 μg/mL), and salvianolic acid B group (200 μg/mL) are 1.67%, and 2.0%, respectively. The cell cytotoxicity linear equations of GMI group and salvianolic acid B group were calculated by using these values. Take GMI and salvianolic acid B group (GMI 20 μg/mL+salvianolic acid B 100 μg/mL) treatment for example (FIG. 2C), the cell cytotoxicity 44.9% is put in y value of GMI cell cytotoxicity linear equation and salvianolic acid B cell cytotoxicity linear equation, respectively, to obtain IC_{x,GMI}=50.79, and IC_{x,SalB}=10818.79. Next, these values are put in the CI formula to obtain CI=(20/50.79)+ (100/10818.79)=0.4. The CI value<1 indicates that the GMI (20 μg/mL) plus salvianolic acid B (100 μg/mL) group has synergism on PCSC cytotoxicity. The same method was used for calculating the CI value of the GMI (20 μg/mL) plus salvianolic acid B (200 μg/mL) group (FIG. 2C), and the value is 0.36. The CI value<1 indicates that the GMI (20 g/mL) plus salvianolic acid B (200 μg/mL) group also has synergism on PCSC cytotoxicity. On the other hand, the CI value of the GMI (100 μg/mL) plus salvianolic acid B (100 μg/mL) group is 0.92; and the CI value of the GMI (100 μg/mL) plus salvianolic acid B (200 μg/mL) group is 0.9. The CI values are both <1, and indicate that compared to GMI treatment only (FIG. 2A), and salvianolic acid B treatment only (FIG. 2B), the GMI plus salvianolic acid B treatment (FIG. 2C) has synergism on PCSC cytotoxicity.

### Example 4

### 4.1 the Synergic Effect of GMI and Salvianolic Acid B on Glioblastoma Cancer Stem Cell

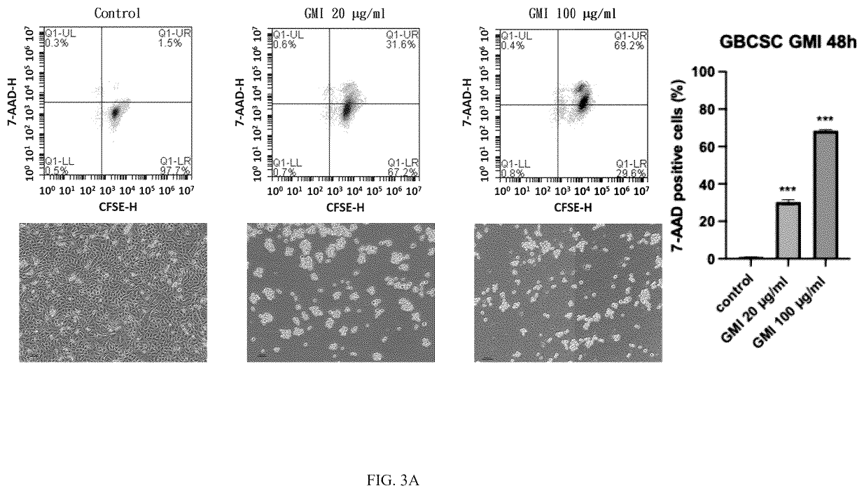

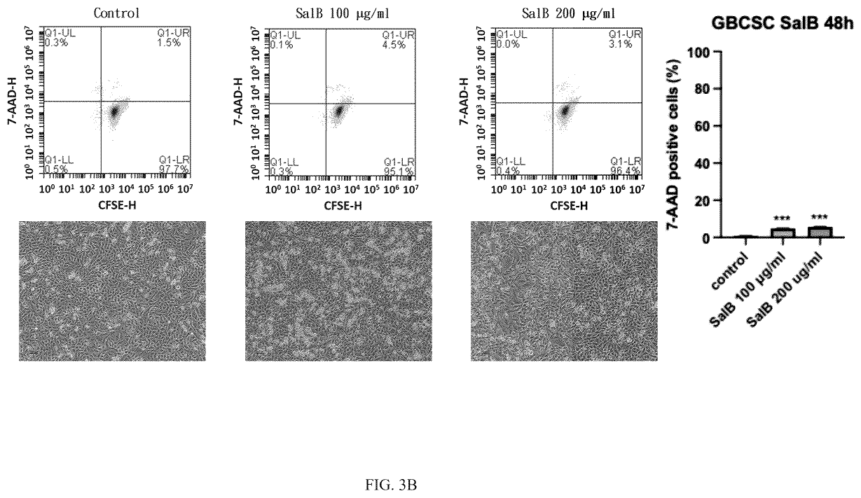

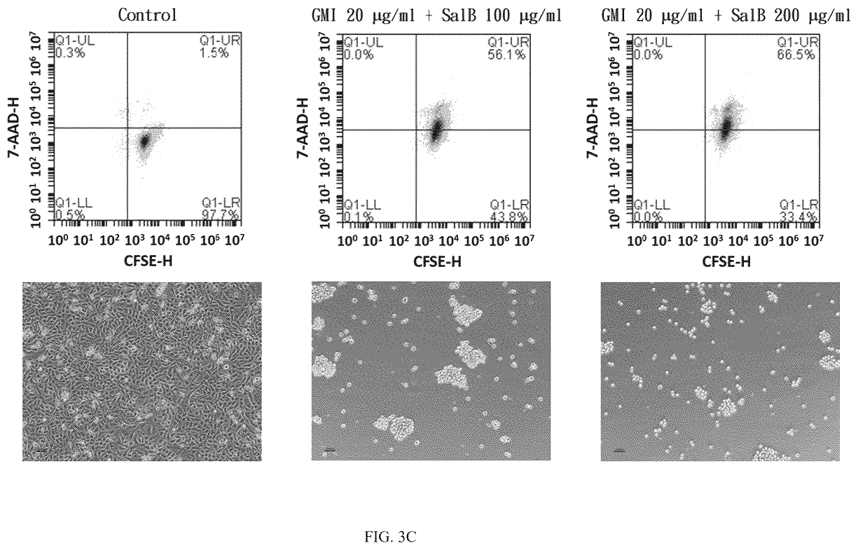

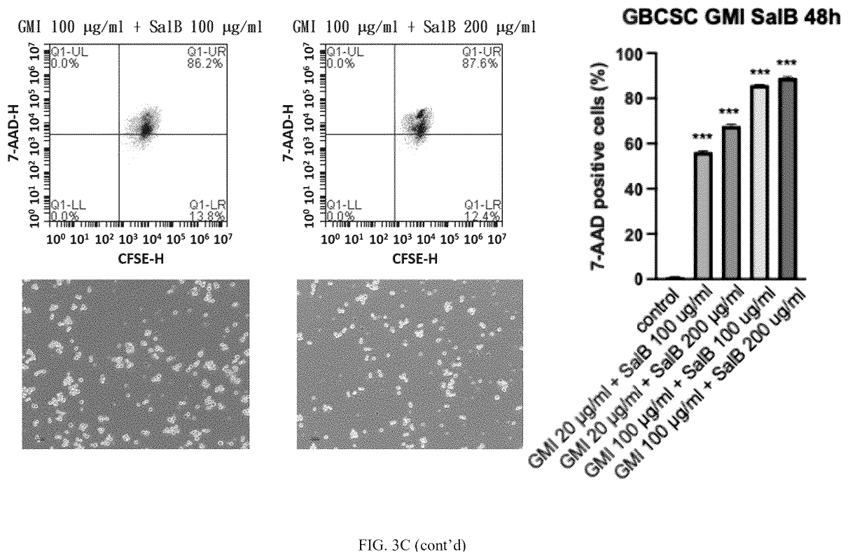

In order to mimic tumor microenvironment, we investigated the cytotoxicity effect of GMI and salvianolic acid B on glioblastoma cancer stem cell (GBCSC) under PBMC existed condition. First, GBCSC (1×10⁵ cells) and PBMC (2×10⁵ cells) were seeded into 24-well plate and treated with different concentration of GMI for 48 hours. After 7-AAD staining, the frequency of GBCSC death was analyzed by flow cytometry. As shown in FIG. 3A, GMI treatment could induce cell death on GBCSC. Although salvianolic acid B could slightly induce cell death of GBCSC, the frequency of cell death is lower than 10% (FIG. 3B). Nevertheless, the sensitivity to GMI in GBCSC was dramatically improved in combination with salvianolic acid B (FIG. 3C). Overall, these findings demonstrated the enhanced anti-GBCSC activity via the synergic effect of GMI and salvianolic acid B.

4.2 the Synergism of GMI and Salvianolic Acid B of this Invention for GBCSC Cytotoxicity

Three different effects of multiple drugs have been proposed, including (1) synergism; (2) additive effect and (3) antagonism. The combination index (CI) published by Chou TC. et al. Quantitative analysis of dose-effect relationships: the combined effects of multiple drugs or enzyme inhibitors, Adv Enzyme Regul, Vol. 22, 1984, pp. 27-55 and Liang Zhao et al. Evaluation of Combination Chemotherapy: Integration of Nonlinear Regression, Curve Shift, Isobologram, and Combination Index Analysis, Clinical Cancer Research, Vol. 10, 2004, pp. 7994-8004 can be used in analyzing the drug-drug interaction. The calculation formula is CI=C_{A,x}/IC_{x,A}+C_{B,x}/IC_{x,B}. Wherein, C_{A,x} and C_{B,x} are the concentrations of drug A and drug B used in combination to achieve x % drug effect. IC_{x,A} and IC_{x,B} are the concentrations for single agents to achieve x % drug effect. CI<1 indicates that the drugs used in combination have synergism. CI=1 indicates that the drugs used in combination have additive effect. CI>1 indicates that the drugs used in combination have antagonism.

In this example, the results of FIGS. 3A-3C which presented the data of the rates of cell cytotoxicity statistics from three independent experiments analyzed by the flow cytometer. As shown in FIG. 3A, the rates of cell cytotoxicity of the control group, GMI group (20 μg/mL), and GMI group (100 μg/mL) are 0.7%, 32%, and 67.8%, respectively. As shown in FIG. 3B, the rates of cell cytotoxicity of the Salvianolic acid B group (100 μg/mL), and salvianolic acid B group (200 μg/mL) are 4.9%, and 5.67%, respectively. The cell cytotoxicity linear equations of GMI group and salvianolic acid B group were calculated by using these values. Take GMI and salvianolic acid B group (GMI 20 μg/mL+salvianolic acid B 100 μg/mL) treatment for example (FIG. 3C), the cell cytotoxicity 56.2% is put in y value of GMI cell cytotoxicity linear equation and salvianolic acid B cell cytotoxicity linear equation, respectively, to obtain IC_{x,GMI}=77.12, and IC_{x,SalB}=2282.87. Next, these values are put in the CI formula to obtain CI=(20/77.12)+ (100/2282.87)=0.3. The CI value<1 indicates that the GMI (20 μg/mL) plus salvianolic acid B (100 μg/mL) group has synergism on GBCSC cytotoxicity. The same method was used for calculating the CI value of the GMI (20 μg/mL) plus salvianolic acid B (200 μg/mL) group (FIG. 3C), and the value is 0.28. The CI value<1 indicates that the GMI (20 μg/mL) plus salvianolic acid B (200 μg/mL) group also has synergism on GBCSC cytotoxicity. On the other hand, the CI value of the GMI (100 μg/mL) plus salvianolic acid B (100 μg/mL) group is 0.83; and the CI value of the GMI (100 μg/mL) plus salvianolic acid B (200 μg/mL) group is 0.82. The CI values are both <1, and indicate that compared to GMI treatment only (FIG. 3A), and salvianolic acid B treatment only (FIG. 3B), the GMI plus salvianolic acid B treatment (FIG. 3C) has synergism on GBCSC cytotoxicity.

While the present disclosure has been described and illustrated with reference to specific embodiments thereof, these descriptions and illustrations are not limiting. It should be understood by those skilled in the art that various changes may be made and equivalents may be substituted without departing from the true spirit and scope of the present disclosure as defined by the appended claims. The illustrations may not be necessarily drawn to scale. There may be distinctions between the artistic renditions in the present disclosure and the actual apparatus due to manufacturing processes and tolerances. There may be other embodiments of the present disclosure which are not specifically illustrated. The specification and drawings are to be regarded as illustrative rather than restrictive. Modifications may be made to adapt a particular situation, material, composition of matter, method, or process to the objective, spirit and scope of the present disclosure. All such modifications are intended to be within the scope of the claims appended hereto. While the methods disclosed herein have been described with reference to particular operations performed in a particular order, it will be understood that these operations may be combined, sub-divided, or re-ordered to form an equivalent method without departing from the teachings of the present disclosure. Accordingly, unless specifically indicated herein, the order and grouping of the operations are not limitations of the present disclosure.

What is claimed is:

1. A pharmaceutical combination, comprising:
Ganoderma immunomodulatory protein (GMI), a recombinant thereof or a fragment thereof; and
salvianolic acid B or a pharmaceutical acceptable salt thereof.

2. The pharmaceutical combination of claim 1, wherein the GMI, a recombinant thereof or a fragment thereof is derived from Ganoderma lucidum, Ganoderma tsugae, Ganoderma microsporum or Ganoderma sinensis.

3. The pharmaceutical combination of claim 1, wherein the GMI, a recombinant thereof or a fragment thereof is derived from Ganoderma microsporum.

4. The pharmaceutical combination of claim 1, wherein the GMI, a recombinant thereof or a fragment thereof comprises an amino acid sequence of SEQ ID NO: 3.

5. The pharmaceutical combination of claim 1, wherein the recombinant of GMI comprises an amino acid sequence of SEQ ID NO: 4.

6. The pharmaceutical combination of claim 1, wherein the fragment of GMI, comprises an amino acid sequence of SEQ ID NO: 1 or 2.

7. The pharmaceutical combination of claim 1, further comprising at least one pharmaceutically acceptable carrier.

8. The pharmaceutical combination of claim 1, which is in the form of a kit of parts for combined administration.

9. The pharmaceutical combination of claim 1, which is in the form of a combination product or a pharmaceutical composition.

10. A method of inhibiting and/or killing treating cancer stem cells in a subject in need thereof, comprising administering the pharmaceutical combination of claim 1 to the subject.

11. The method of claim 10, wherein the cancer stem cells are selected from the group consisting of pancreatic cancer stem cells and glioblastoma cancer stem cells.

12. The method of claim 11, wherein the pancreatic cancer stem cells are pancreatic cancer cells expressing at least one of CD133, CD24, CD44, DCLK1, CXCR4, ESA, Oct4, and ABCB1.

13. The method of claim 11, wherein the glioblastoma cancer stem cells are glioblastoma cancer cells expressing at least one of CD133, CD90, CD44, L1CAM, A2B5, and GPD1.

14. The method of claim 10, wherein the GMI, a recombinant thereof or a fragment thereof and salvianolic acid B or a pharmaceutical acceptable salt thereof are administered jointly or independently at the same time.

15. The method of claim 10, wherein the GMI, a recombinant thereof or a fragment thereof and salvianolic acid B or a pharmaceutical acceptable salt thereof are administered separately within time intervals.

16. A method of treating and/or preventing cancers associated with CSCs cancer or treating cancer that is metastatic, refractory to a chemotherapy or radiotherapy, inherently resistant to chemotherapy or has relapsed in the subject after an initial treatment in a subject in need thereof, comprising administering the pharmaceutical combination of claim 1 to the subject.

17. The method of claim 16, wherein the cancer is selected from breast cancers, head and neck cancer, lung cancer, ovarian cancer, pancreatic cancer, colorectal carcinoma, prostate cancer, liver cancer, melanoma, multiple myeloma, brain tumor, sarcoma, medulloblastoma, glioblastoma, leukemia, and metastatic, refractory or relapsed of any of the above cancer.

18. The method of claim 17, wherein the pancreatic cancer comprises pancreatic cancer cells expressing at least one of CD133, CD24, CD44, DCLK1, CXCR4, ESA, Oct4 and ABCB1.

19. The method of claim 17, wherein the glioblastoma cancer comprises glioblastoma cancer cells expressing at least one of CD133, CD90, CD44, L1CAM, A2B5, and GPD1.

20. The method of claim 16, wherein the GMI, a recombinant thereof or a fragment thereof and salvianolic acid B or a pharmaceutical acceptable salt thereof are administered jointly or independently at the same time.

21. The method of claim 16, wherein the GMI, a recombinant thereof or a fragment thereof and salvianolic acid B or a pharmaceutical acceptable salt thereof are administered separately within time intervals.


SEQUENCE LISTING (4 sequences)

SEQ ID NO: 1 - protein, 6 aa, synthetic construct
LAWNVK

SEQ ID NO: 2 - protein, 10 aa, synthetic construct
DLGVRPSYAV

SEQ ID NO: 3 - protein, 111 aa, synthetic construct
MSDTALIFTLAWNVKQLAFDYTPNWGRGRPSSFIDTVTFPTVLTDKAYTYRVVVSGKDLGVRPSYAVESDGSQKINFLEYNSGYGIADTNTIQVYVIDPDTGNNFIVAQWN

SEQ ID NO: 4 - protein, 140 aa, synthetic construct
EAEAEFMSDTALIFTLAWNVKQLAFDYTPNWGRGRPSSFIDTVTFPTVLTDKAYTYRVVVSGKDLGVRPSYAVESDGSQKINFLEYNSGYGIADTNTIQVYVIDPDTGNNFIVAQWNYLEQKLISEEDLNSAVDHHHHHH